(19) United States
(12) Patent Application Publication
(10) Pub. No.: US 2012/0191154 A1
(43) Pub. Date: Jul. 26, 2012

(54) System and Method for ATP Treatment Utilizing Multi-Electrode Left Ventricular Lead

(75) Inventors: Kyungmoo Ryu, Palmdale, CA (US); Stuart Rosenberg, Castaic, CA (US); Allen Keel, San Francisco, CA (US); Taraneh Ghaffari Farazi, Santa Clara, CA (US); Richard Williamson, Los Angeles, CA (US); Mark Carlson, Calabasas, CA (US)

(73) Assignee: PACESETTER, INC., Sylmar, CA (US)

(21) Appl. No.: 13/012,574

(22) Filed: Jan. 24, 2011

(51) Int. Cl. A61N 1/365

(52) U.S. Cl. 607/14

(57) ABSTRACT

An implantable medical device includes a lead configured to be located proximate to the left ventricle (LV) of the heart, the lead including multiple LV electrodes to sense cardiac activity at multiple LV sensing sites. The a detection module to detect an arrhythmia that represents at least one of a tachycardia and fibrillation based at least in part on the cardiac activity sensed at the multiple LV sensing sites. The ATP therapy module to identify at least one of an ATP configuration or an ATP therapy site based on the cardiac sensed activity at the LV sensing sites, the ATP therapy module to control delivery of antitachycardia pacing (ATP) therapy at the ATP therapy site. The ATP therapy module delivers a stimulus to electrodes at one or more of an LV site, right ventricular (RV) site and right atrial (RA) site, the detection module to sense evoked responses at the LV sensing sites, the ATP therapy module to designate the ATP therapy site to include at least the LV sensing site with a shortest activation time relative to the one or more LV site, RV site and RA site where the stimulus is delivered.

### BACKGROUND OF THE INVENTION

Embodiments generally relate to methods and systems to detect and treat tachycardia, such as through the use of anti-tachycardia pacing.

Numerous types of devices and systems exist today that monitor and treat abnormal behavior of the heart (arrhythmias). Examples of arrhythmias include tachycardia, fibrillation and the like. With normal conduction, the cardiac contractions are very organized and timed so that the top chambers (the atria) contract before the lower chambers and the heart rate is maintained between 60 and 120 beats per minute. Fast, abnormal heart rhythms are called tachyarrhythmias.

Ventricular tachycardia (VT) is a tachyarrhythmia that originates in the ventricle and may be life-threatening. Symptoms of VT include feeling faint, sometimes passing out, dizziness, or a pounding in the chest.

Tachycardias can result due to any number of reasons. For example, patients who have had myocardial infarctions, or other diseases that create scarring in the ventricular region of the heart, often develop monomorphic ventricular tachycardias. A monomorphic ventricular tachycardia (MVT) is a type of tachycardia that originates from one ventricular focus. These tachycardias often arise in and around an area of scarring on the heart. They are typically uniform and typically occur at a regular rate. Faster MVTs are often associated with hemodynamic compromise, whereas slower MVTs can be very stable.

Ventricular tachycardia may be treated with medication, catheter ablation, surgery, and an implantable cardioverter defibrillator (ICD). The ICD treats ventricular tachycardia by pacing the heart (antitachycardia pacing, ATP) or delivering a high voltage shock to terminate the arrhythmia.

Conventional ATP is not always successful. Approaches to improve ATP efficacy are desirable.

Recently, it has been proposed to utilize bi-ventricular (BV) ATP rather than right ventricular (RV) pacing alone to terminate VT. As proposed, if the first pacing configuration were unsuccessful, ATP would be performed using a different pacing configuration. Exemplary pacing configurations include, but are not limited to, pacing only the RV, pacing only the left ventricle (LV), and pacing the LV and the RV (e.g., BV ATP) simultaneously.

A need remains to improve the likelihood of capture and efficacy of pace termination of arrhythmias. A need also remains to improve characterization and understanding of the reentrant VT circuit dynamics.

### SUMMARY

In accordance with some embodiments described herein, methods and systems are provided that provide a multi-electrode LV lead to form multiple LV sensing sites that are utilized, among other things, to determine mechanisms of VT maintenance (e.g., a site or sites of ectopic activity causing reentrant tachycardia) and/or to characterize a reentrant activation pattern. In accordance with some embodiments described herein, methods and systems are provided for detecting polymorphic tachycardia and for using mass ATP therapy for stimulation through multiple ventricular sites to achieve termination. The sites at which the ATP therapies are delivered may be guided by the determination of the mechanism maintaining VT and/or the reentrant activation pattern. In accordance with embodiments described herein, methods and systems are provided for detecting ventricular fibrillation and using mass stimulation through multiple ventricular sites to terminate the fibrillation.

In an embodiment, ATP schemes may be attempted and then progressively moved to more aggressive ATP therapies for the purpose of alleviating a VT. The movement from one ATP scheme to a more aggressive scheme may involve increasing or decreasing the pacing cycle length, increasing the number of therapy deliver sites, increasing the number of ATP pulses and the like. Optionally, capture confirmation may be determined during delivery of an ATP therapy in order to identify and demonstrate whether the ATP therapy is appropriate or inappropriate to entrain a particular type of VT activation pattern. Following the capture confirmation, methods and systems are described herein to provide more or less aggressive ATP schemes. Optionally, VT characterization may be performed using one or more of multiple activation analysis processes. For example, post pacing intervals (PPI) may be determined and utilized for multiple LV electrodes in connection with characterization of a reentrant activation pattern. In accordance with embodiments described herein, methods and systems are provided by which an ATP therapy may be preceded by prepulses, such as delivered from any electrode or electrodes on the multi-electrode LV lead. Optionally, combinations of simultaneous and sequential ATP pacing therapies may be delivered from a multi-site LV electrode. For example, electrode sets may be used to simultaneously deliver ATP therapies that are sequentially followed by entirely separate or partially overlapping electrode sets which deliver a separate ATP therapy.

In accordance with one embodiment, an implantable medical device IMD is provided that comprises a lead configured to be located proximate to the LV of the heart. The lead includes multiple LV electrodes to sense cardiac activity at multiple LV sensing sites. The device includes a detection module to detect an arrhythmia that represents at least one of a tachycardia and fibrillation and an ATP therapy module. The ATP therapy module identifies at least one of an ATP configuration or an ATP therapy site based on a relation of the cardiac activity between the LV sensing sites. The ATP therapy module controls delivery of ATP therapy at the ATP therapy site.

Optionally, the ATP therapy module delivers a stimulus to electrodes at one or more; an LV site and RV site. The detection module senses evoked responses at the LV sensing sites. The ATP therapy module designates the ATP therapy site to include at least the LV sensing site with a shortest activation time relative to the one or more LV site and RV site. Optionally, the ATP therapy module delivers a stimulus to the heart and measures PPI between the stimulus site and the LV sensing sites. The ATP therapy module may designate the ATP therapy site to include the LV sensing site having a shortest PPI. Optionally, the ATP therapy module delivers a stimulus to the heart and obtains electrograms associated with the LV sensing sites. The ATP therapy module determines a degree of fractionation of the electrograms. The ATP therapy module designates at least one of ATP configuration and the ATP therapy site to include the LV sensing site corresponding to a least degree of fractionation.

Optionally, the device comprises an impedance measuring circuit to measure impedance associated with the LV electrodes during normal sinus rhythm or a pacing therapy. The ATP therapy module may designate the ATP therapy site to correspond to the LV sensing site having minimum electrode impedance. Optionally, the ATP therapy module determines an activation pattern of activation times corresponding to the LV sensing sites. The ATP therapy module may designate the ATP therapy site to be the LV sensing site with an earliest activation time. The ATP therapy module may determine an activation pattern for a VT reentrant circuit. The ATP therapy module may designate the ATP therapy site to be the LV electrode proximate to a starting site of the VT reentrant circuit. Optionally, the ATP therapy module may designate at least one ATP therapy site based on one or more of an activation time, post pacing interval, waveform morphology, electrode impedance, or activation pattern.

In accordance with an alternative embodiment, a method is provided for controlling ATP. The method comprises sensing signals from a lead including multiple LV electrodes representative of cardiac activity at multiple LV sensing sites; detecting an arrhythmia that represents at least one of a tachycardia and fibrillation; identifying at least one of an ATP configuration or an ATP therapy site based on a relation between the cardiac activity at the LV sensing sites; and controlling delivery of ATP therapy at the ATP therapy site.

### BRIEF DESCRIPTION OF THE DRAWINGS

FIG. 1A illustrates a simplified diagram of an implantable medical device for delivering multi-chamber stimulation in accordance with an embodiment.

FIG. 1B illustrates a functional block diagram of the multi-chamber implantable medical device of FIG. 1 in accordance with an embodiment.

FIG. 2A illustrates a lead configuration utilized in accordance with an embodiment.

FIG. 2B illustrates a process carried out for delivering an ATP therapy in accordance with an embodiment.

FIG. 3A illustrates a flowchart for a method to characterize a VT or VF activation pattern based on an electrical activation sequence analysis in accordance with one embodiment.

FIGS. 3B and 3C illustrate examples of activation timings that may correspond to sensed events of interest in accordance with an embodiment.

FIG. 3D illustrates examples of different activation patterns associated with different ectopys in accordance with an embodiment.

FIG. 4A illustrates a flowchart that utilizes a capture analysis to characterize VT activation patterns in accordance with an alternative embodiment.

FIG. 4B illustrates a flow chart that utilizes wave morphology analysis to characterize VT activation patterns in accordance with an alternative embodiment.

FIG. 5 illustrates a flowchart that utilizes a PPI analysis to characterize VT activation patterns in accordance with an alternative embodiment.

FIG. 6 illustrates a flow chart that utilizes electrograms to characterize VT activation patterns in accordance with an alternative embodiment.

FIG. 7 illustrates a flow chart of a method that selects parameters for the ATP therapy in accordance with an embodiment.

FIG. 8 illustrates examples of alternative pacing pulse morphologies in accordance with an embodiment.

FIG. 9 illustrates a graphical example of the manner in which ATP therapy electrodes and ATP capture confirmation may be performed in accordance with an embodiment.

### DETAILED DESCRIPTION

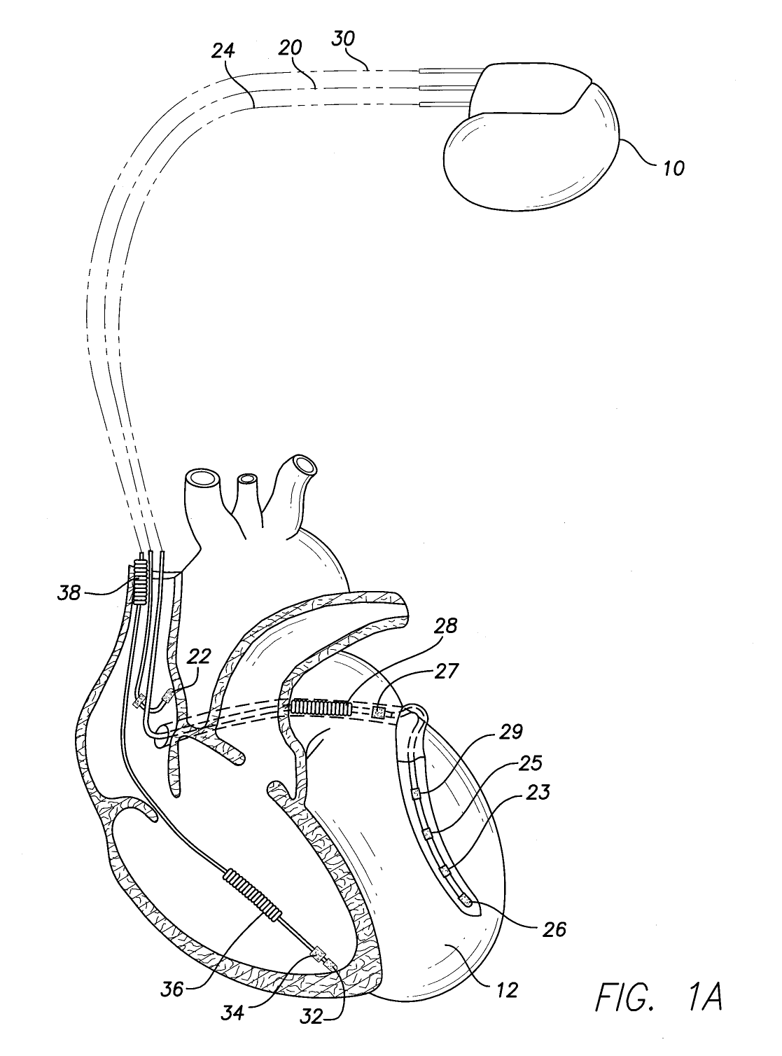

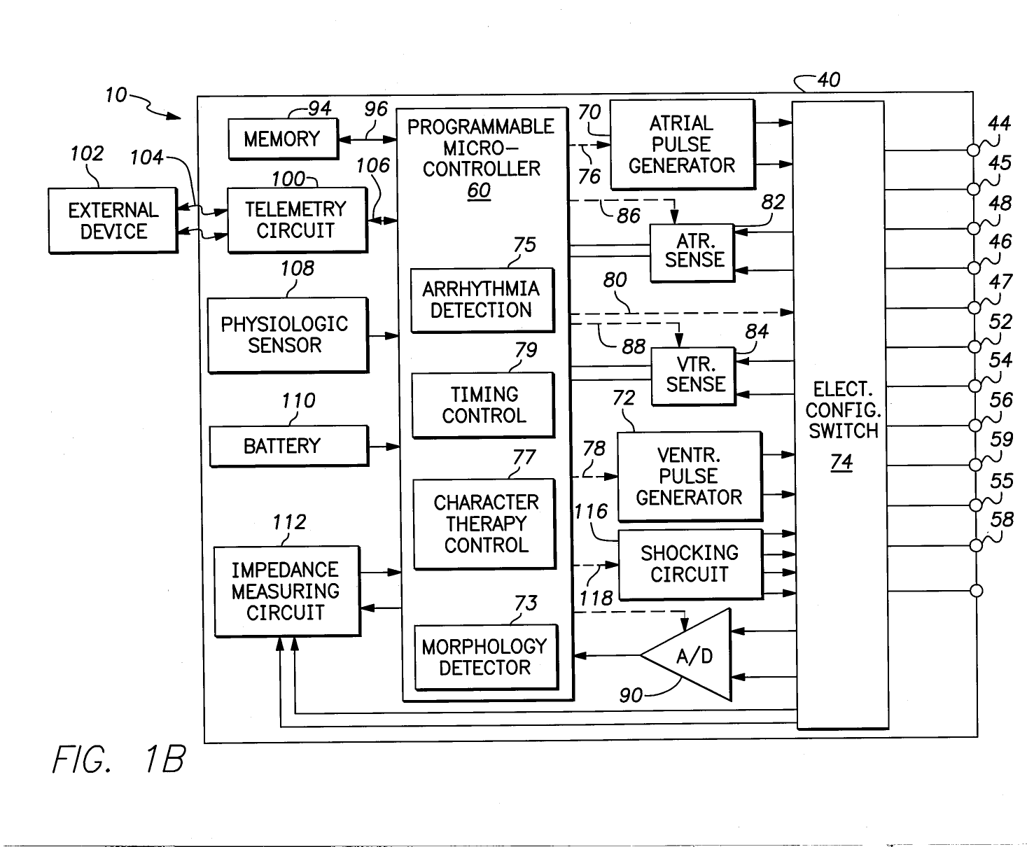

FIG. 1A illustrates a simplified diagram of an implantable medical device (IMD) 10 in electrical communication with at least three leads 20, 24 and 30 implanted in or proximate a patient's heart 12 for delivering multi-chamber stimulation (e.g. pacing, ATP therapy, high voltage shocks and the like) according to an embodiment. As explained below, the leads 20, 24 and 30 are used to sense VT and ventricular fibrillation (VF) and to deliver, among other things, ATP therapies. The device 10 is programmable, by an operator, to set certain operating parameters, as well as therapy-related parameters. The device 10 is configured to operate with various configurations of leads. Exemplary lead configurations are shown in the Figures. The device 10 is configured to deliver various types of ATP therapies.

To sense atrial cardiac signals and to provide right atrial (RA) chamber stimulation therapy, the device 10 is coupled to an implantable RA lead 20 having at least an atrial tip electrode 22, which typically is implanted in the patient's RA appendage. The device 10 may be a pacing device, a pacing apparatus, a cardiac rhythm management device, an implantable cardiac stimulation device, an implantable cardioverter/defibrillator (ICD) and/or a cardiac resynchronization therapy (CRT) device.

To sense left atrial (LA) and ventricular cardiac signals and to provide left chamber pacing therapy, the device 10 is coupled to an LV lead 24. The LV lead 24 may receive atrial and ventricular cardiac signals and deliver LV pacing therapy using an LV tip electrode 26, and intermediate LV electrodes 23, 25 and 29. LA pacing therapy uses, for example, first and second LA electrodes 27 and 28. The LV and LA electrodes 23-29 may represent sensing sites, where cardiac signals are sensed, and/or may represent ATP therapy sites. An RV lead 30 includes an RV tip electrode 32, an RV ring electrode 34, an RV coil electrode 36, and a superior vena cava (SVC) coil electrode 38 (also known as an RA coil electrode). The RV lead 30 is capable of sensing cardiac signals, and delivering stimulation in the form of pacing and shock therapy to the SVC and/or RV.

Embodiments are described herein, whereby multiple LV electrodes are utilized to sense cardiac activity at multiple LV sensing sites, alone or in combination with RV, RA and LA sensing sites, in a manner that affords improved characterization and understanding of VT or VF circuit dynamics. Information collected at the sensing sites (e.g., LV alone or LV and RV, RA and/or LA) is utilized to determine sites, at which ATP therapy should be delivered. The information may also be used to determine an ATP configuration. In certain embodiments, multiple LV electrodes are utilized to deliver ATP pulses at one or more LV sites and along one or more therapy vectors. In certain embodiments, the ATP configuration includes ATP therapy delivered only from multiple LV sites, or ATP therapy delivered from multi-site LV BV ATP. Multi-site LV ATP will produce relatively uniform resulting activation propagation across the heart, as well as capture a relatively large area of the heart. A likelihood of entrainment of a reentrant VT circuit is dependent in part on an amount of the heart mass that is captured and upon the propagation of an ATP therapy across the heart.

In accordance with certain embodiments, multi-site LV sensing is utilized to analyze reentrant activation of a VT episode, confirm entrainment/capture of the VT episode and/or determine a degree of entrainment/capture of the VT episode. For example, the analysis of multi-site LV sensing may be utilized to determine ATP therapy sites and/or ATP configurations.

Optionally, multi-site LV sensing and/or multi-site LV therapy may be utilized to deliver an ATP therapy to terminate VF. Successful conversion of VF is based in part on capturing or resetting a certain mass of the ventricles that are fibrillating (generally referred to as a critical mass). When the left ventricle experiences fibrillation, multi-site LV ATP (without RV ATP) or BV ATP (with single site LV or multi-site LV) captures a large area of the fibrillating ventricle(s). The LV ATP or BV ATP exhibits high efficacy in treating VF by entraining a VF driver without shocking at energy levels higher than pacing pulses.

FIG. 1B illustrates a block diagram of the stimulation device 10, which is capable of treating both fast and slow arrhythmias with stimulation therapy, including cardioversion, defibrillation, and pacing stimulation. While a particular multi-chamber device is shown, this is for illustration purposes only. It is understood that the appropriate circuitry could be duplicated, eliminated or disabled in any desired combination to provide a device capable of treating the appropriate chamber(s) with cardioversion, defibrillation and pacing stimulation.

The housing 40 for the stimulation device 10, shown schematically in FIG. 1B, is often referred to as the “can”, “case” or “case electrode” and may be programmably selected to act as the return electrode for some or all “unipolar” modes. The housing 40 may further be used as a return electrode alone or in combination with one or more of the electrodes, 28, 36 and 38 of FIG. 1, for shocking purposes. The housing 40 further includes a connector (not shown) having a plurality of terminals, 44, 45, 46, 47, 48, 52, 54, 55, 56, 58, and 59. To achieve sensing, pacing and shocking in desired chambers of the heart, the terminals 44-59 are connected to corresponding combinations of electrodes 22-36.

An electrode configuration switch 74 connects the sensing electronics to the desired ones of the terminals 44-59 of corresponding sensing electrodes. For example, terminals 55-59 may be coupled to LV electrodes 23, 25, 26 and 29. The switch 74 may connect terminals 55-59 to one or more ventricular sensing circuits 84, which provides signals, representative of cardiac activity, to the microcontroller. The circuit 84 may amplify, filter, digitize and/or otherwise process the sensed signals from the LV electrodes 23, 25, 26 and 29. The circuit 84 may provide separate, combined or difference signals to the microcontroller 60 representative of the sensed signals form the LV electrodes 23, 25, 26 and 29. The circuit 84 may also receive sensed signals from RV electrodes. The atrial sensing circuit 82 is connected through the switch 74 to desired RA and/or LA electrodes to sense RA and/or LA cardiac activity.

The stimulation device 10 includes a programmable microcontroller 60 that controls the various modes of stimulation therapy. The microcontroller 60 includes a microprocessor, or equivalent control circuitry, designed specifically for controlling the delivery of stimulation therapy and may further include RAM or ROM memory, logic and timing circuitry, state machine circuitry, and I/O circuitry. The microcontroller 60 includes the ability to process or monitor input signals (data) as controlled by a program code stored in memory. The details of the design and operation of the microcontroller 60 are not critical to the present invention. Rather, any suitable microcontroller 60 may be used.

The microcontroller 60 includes an arrhythmia detection module 75 that analyzes sensed signals and determines when an arrhythmia is occurring. The detection module 75 receives signals sensed by electrodes located at sensing sites. For example, the signals may be received from multiple LV electrodes which represent cardiac activity at the corresponding multiple LV sensing sites. The detection module 75 detects an arrhythmia that represents at least one of a tachycardia and fibrillation, such as VT and VF.

The microcontroller 60 performs characterization of VT and/or VF activation patterns in accordance with various embodiments described herein. The detection module 75 may perform a VT capture analysis to characterize VT activation patterns in accordance with an embodiment. The detection module 75 may analyze electrograms to characterize. VT activation patterns in accordance with an alternative embodiment.

The microcontroller 60 includes a characterization and therapy control module 77 that performs multiple functions. The control module 77 controls delivery of one or more pacing pulses and interacts with the modules 73 and 75 in connection with processes described herein to characterize VT/VF activation patterns. For example, the control module 77 may deliver one or more pacing pulses in connection with performing an electrical activation sequence analysis to characterize VT activation patterns. The control module 77 also identifies the type of ATP therapies to be used, and controls delivery of the ATP therapies. For example, the control module 77 identifies at least one of an ATP configuration or an ATP therapy site based on a relation of the cardiac activity between the LV sensing sites. The control module 77 controls delivery of ATP therapy at the ATP therapy site. For example, the control module 77 may deliver a stimulus to electrodes at one or more of an LV site and RV site. The detection module 75 senses evoked responses at the LV sensing sites. The control module 77 designates the ATP therapy site to include at least the LV sensing site with a shortest activation time relative to the one or more LV site and RV site.

When using PPI based characterization, the control module 77 delivers a stimulus at a stimulus site to the heart and the detection module 75 measures PPI between the stimulus site and the LV sensing sites. The detection module 75 performs PPI analysis to characterize VT activation patterns in accordance with an alternative embodiment. The control module 77 designates the ATP therapy site to include the LV sensing site having a shortest PPI.

The microcontroller 60 includes a morphology detection module 73 that may perform wave morphology analysis to characterize VT activation patterns in accordance with an alternative embodiment. When using waveform morphology based characterization, the control module 77 delivers a stimulus to the heart and the morphology detection module 73 obtains electrograms associated with the LV sensing sites. The morphology detection module 73 may determine a degree of fractionation of the electrograms. The control module 77 designates at least one of ATP configuration and the ATP therapy site to include the LV sensing site corresponding to a least degree of fractionation. The degree of fractionation is assessed based on at least one of a number of deflections in the electrograms, a number of peaks in the electrograms, a width of the electrograms, and an area under the electrograms and a fast fourier transform (FFT) of the electrograms.

When using impedance based characterization, the impedance measuring circuit 112 measures impedance associated with the LV electrodes during normal sinus rhythm or a pacing therapy. For example, the impedance measuring circuit 112 may measure a pacing threshold associated with one or more LV electrodes and/or one or more vectors. The control module 77 may then designate the ATP therapy site to correspond to the LV sensing site or vector having a minimum electrode impedance, such as the lowest pacing threshold.

When using activation pattern based characterization, the control module 77 determines an activation pattern of activation times corresponding to the LV sensing sites. The control module 77 designates the ATP therapy site to be the LV sensing site with an earliest activation time. For example, the control module 77 may determine an activation pattern for a VT reentrant circuit. The control module 77 designates the ATP therapy site to be the LV electrode proximate to a starting site of the VT reentrant circuit.

The control module 77 may designate at least one ATP therapy site based on at least one of an activation time, PPI, waveform morphology, electrode impedance, or activation pattern. For example, the control module may designate an ATP cycle length to correspond to a predetermined percentage of a VT cycle length. The control module may change at least one of a stimulus voltage and pulse width for subsequent ATP therapies when a prior attempted ATP therapy is not successful in converting an arrhythmia to a normal sinus rhythm.

As shown in FIG. 1 B, an atrial pulse generator 70 and a ventricular pulse generator 72 generate pacing and ATP stimulation pulses for delivery by desired electrodes. The electrode configuration switch 74 (also referred to as switch bank 74) controls which terminals 44-60 receive one or more pulses of an ATP therapy atrial ventricular pulse generators 70 and 72. The atrial and ventricular pulse generators, 70 and 72, may include dedicated, independent pulse generators, multiplexed pulse generators, shared pulse generators or a single common pulse generator. The pulse generators 70 and 72 are controlled by the microcontroller 60 via appropriate control signals 76 and 78, respectively, to trigger or inhibit stimulation pulses. The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, PVARP intervals, noise detection windows, evoked response windows, alert intervals, marker channel timing, etc.

The switch bank 74 includes a plurality of switches for connecting the desired electrodes to the appropriate I/O circuits, thereby providing complete electrode programmability. The switch 74, in response to a control signal 80 from the microcontroller 60, determines the polarity of the stimulation pulses (e.g., unipolar, bipolar, co-bipolar, etc.) by selectively closing the appropriate combination of switches (not specifically shown). Atrial sensing circuits 82 and ventricular sensing circuits 84 may also be selectively coupled to the right atrial lead 20, LV lead 24, and the right ventricular lead 30, through the switch 74 for detecting the presence of cardiac activity in each of the four chambers of the heart. The switch 74 determines the “sensing polarity” of the cardiac signal by selectively closing the appropriate switches. The outputs of the atrial and ventricular sensing circuits 82 and 84 are connected to the microcontroller 60 which, in turn, is able to trigger or inhibit the atrial and ventricular pulse generators 70 and 72, respectively. The sensing circuits 82 and 84, in turn, receive control signals over signal lines 86 and 88 from the microcontroller 60 for purposes of controlling the gain, threshold, the polarization charge removal circuitry (not shown), and the timing of any blocking circuitry (not shown) coupled to the inputs of the sensing circuits,82 and 86.

For arrhythmia detection, the device 10 utilizes the atrial and ventricular sensing circuits 82 and 84 to sense cardiac signals and the arrhythmia detection module 75 to determine whether a rhythm is physiologic or pathologic. As used herein “sensing” is the receipt or noting of an electrical signal, and “detection” is the processing of these sensed signals and determining the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 60 by comparing them to a predefined rate zone limit (e.g., bradycardia, normal, low rate VT, high rate VT, and fibrillation rate zones) and/or various other characteristics (e.g., sudden onset, stability, physiologic sensors, morphology, etc.) in order to determine the type of remedial therapy that is needed (e.g., bradycardia pacing, ATP, cardioversion shocks or defibrillation shocks, collectively referred to as “tiered therapy”).

Cardiac signals are also applied to the inputs of an analog-to-digital (A/D) data acquisition system 90. The data acquisition system 90 is configured to acquire intracardiac electrogram signals, convert the raw analog data into a digital signal, and store the digital signals for later processing and/or telemetric transmission to an external device 102. The data acquisition system 90 samples cardiac signals across any pair of desired electrodes. The data acquisition system 90 may be coupled to the microcontroller 60, or other detection circuitry, for detecting an evoked response from the heart 12 in response to an applied stimulus, thereby aiding in the detection of “capture.” Capture occurs when an electrical stimulus applied to the heart is of sufficient energy to depolarize the cardiac tissue, thereby causing the heart muscle to contract.

The microcontroller 60 is further coupled to a memory 94 by a suitable data/address bus 96, wherein the programmable operating and therapy-related parameters used by the microcontroller 60 are stored and modified, as required, in order to customize the operation of the stimulation device 10 to suit the needs of a particular patient. The operating and therapy-related parameters define, for example, pacing pulse amplitude, pulse duration, electrode polarity, rate, sensitivity, automatic features, arrhythmia detection criteria, and the amplitude, wave shape and vector of each stimulating pulse to be delivered to the patient's heart 12 within each respective tier of therapy.

The operating and therapy-related parameters may be non-invasively programmed into the memory 94 through a telemetry circuit 100 in telemetric communication with the external device 102, such as a programmer, trans-telephonic transceiver, or a diagnostic system analyzer. The telemetry circuit 100 is activated by the microcontroller 60 by a control signal 106. The telemetry circuit 100 advantageously allows intracardiac electrograms and status information relating to the operation of the device 10 (as contained in the microcontroller 60 or memory 94) to be sent to the external device 102 through an established communication link 104.

The stimulation device 10 may include a physiologic sensor 108 to adjust pacing stimulation rate according to the exercise state of the patient. The physiological sensor 108 may further be used to detect changes in cardiac output, changes in the physiological condition of the heart, or diurnal changes in activity (e.g., detecting sleep and wake states). The microcontroller 60 responds by adjusting the various pacing parameters (such as pacing rate, AV Delay, V-V Delay, etc.) at which the atrial and ventricular pulse generators, 70 and 72, generate stimulation pulses.

The battery 110 provides operating power to all of the circuits shown in FIG. 1B. An impedance measuring circuit 112 monitors lead impedance during the acute and chronic phases for proper lead positioning or dislodgement; detects operable electrodes and automatically switches to an operable pair if dislodgement occurs; measures respiration or minute ventilation; measures thoracic impedance for determining shock thresholds; detects when the device has been implanted; measures stroke volume; and detects the opening of heart valves, etc.

The microcontroller 60 further controls a shocking circuit 116 by way of a control signal 118. The shocking circuit 116 generates stimulating pulses of low (up to 0.5 joules), moderate (0.5-10 joules), or high energy (11 to 40 joules), as controlled by the microcontroller 60. Stimulating pulses are applied to the patient's heart 12 through at least two shocking electrodes, and as shown in this embodiment, selected from the LA coil electrode 29, the RV coil electrode 36 the SVC coil electrode 38 and/or the housing 40.

Before further explaining embodiments of the present invention, it is helpful to briefly review the basic electro-physiologic mechanisms responsible for VTs. During the normal cardiac cycle, a cardiac cell membrane depolarizes and repolarizes in a characteristic fashion known as the action potential. Action potential propagation occurs when depolarization in one cell generates current to neighboring cells, forcing membrane sodium channels to open and allowing a rapid excitatory influx of sodium that further depolarizes the membrane. Sodium channels then close. Other ionic currents repolarize the membrane to its resting state over a slow time course that is sufficiently long for sodium channels to recover excitability. Heart rate is important in this process because the interval between recovery in one cycle and activation in the next provides time for the cell to achieve ionic, metabolic and energetic equilibrium.

When cells die in a myocardial infarct, they electrically uncouple from neighboring viable cells, making the infarct completely inexcitable. Intrinsic or paced wavefronts encountering such an obstacle generally split into two components that collide and recombine on the opposite side of the infarct. When tissue adjacent to the infarct excites prematurely, however, reentry can result if one of the wavefronts blocks in a region with reduced excitability, i.e. incomplete sodium channel opening. The reduced excitability can result from in homogeneities in membrane properties, geometric changes that increase the wavefronts electrical load, or incomplete recovery of excitability during a short interval. When blocking of one wavefront occurs, the other wavefront may be able to reenter the initial blocked site, causing what is known as a “reentrant circuit.”

The reentrant circuit can be thought of as a conduction wavefront propagating along a tissue mass of somewhat circular geometry. This circular conduction will consist of a portion of refractory tissue and a portion of excitable tissue. To terminate the circuit, a pacing pulse should be provided at the time and location when the tissue just comes out of refractoriness. If this occurs, the paced activation wavefront proceeds toward the advancing wavefront of the circuit, colliding with the wavefront and interrupting the circuit. If the pacing pulse arrives too soon it will be ineffective because the tissue will still be in refractoriness. If the stimulus arrives too late, it will generate wavefronts both towards the advancing wavefront and towards the tail of the circuit. Although one paced activation wavefront will collide with the advancing wavefront of the reentrant circuit and will halt is progress, the latter paced activation wavefront will act to sustain the reentrant circuit.

Accordingly, the success of ATP therapy in terminating the tachycardia is related to the ability of the paced activation wavefront to arrive at the location of the reentrant circuit in such a manner that the reentrant circuit is modified or interrupted. Factors influencing this process include the distance of the ATP electrode(s) from the reentrant circuit, the ATP stimulus energy, and the timing of the ATP stimuli relative to the conduction velocities and refractory periods of the myocardium.

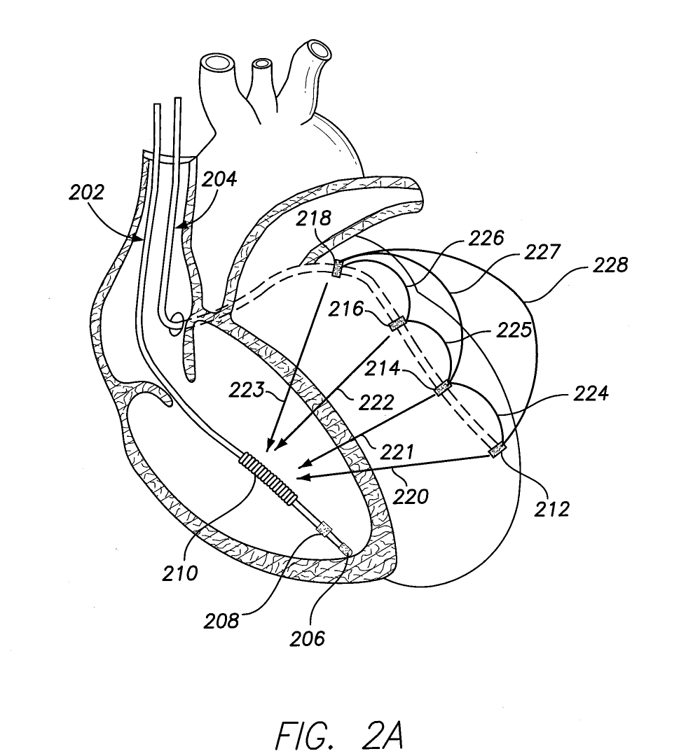

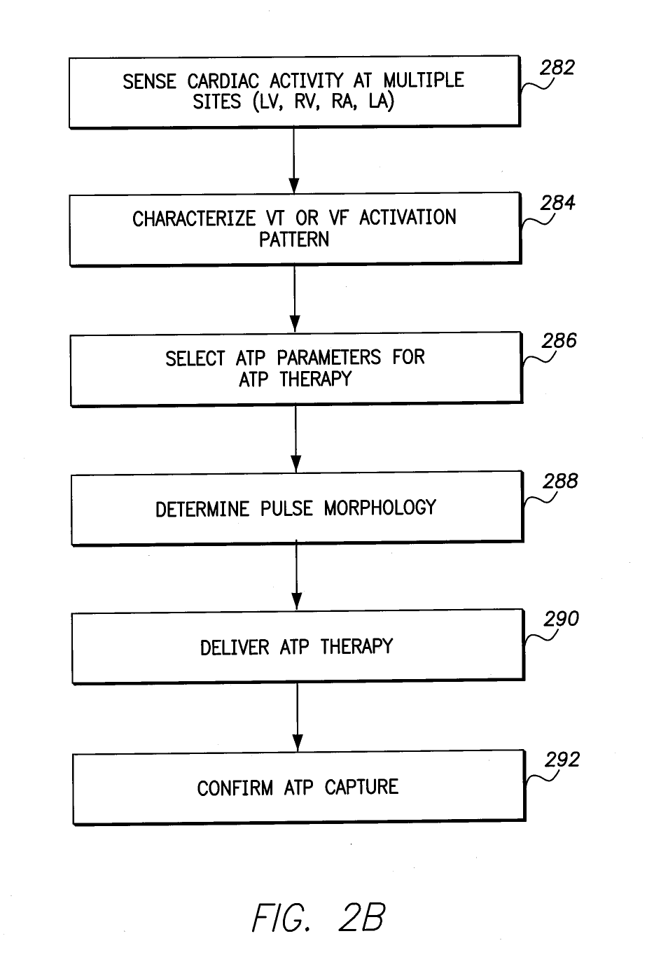

FIG. 2A illustrates RV and LV leads utilized in accordance with an embodiment. In FIG. 2A, an RV lead 202 and an LV lead 204 are shown. The RV lead 202 includes tip, ring and coil RV electrodes 206, 208 and 210 located in the RV. The RV lead 202 may also include an electrode in the RA and/or at the SVC. The LV lead 204 includes a tip LV electrode 212 and LV electrodes 214, 216 and 218. The LV electrodes 214, 216 and 218 are spaced apart from one another along the lateral wall of the LV. The LV lead 204 may include electrodes proximate to the LA.

Optionally, more or fewer LV electrodes may be utilized. Optionally, the LV electrodes may be separated more or positioned closer to one another. Optionally, all or a portion of the LV electrodes may be shifted along the LV lead 204 until positioned proximate to the mitral valve, aortic valve, or the LA ports to/from the pulmonary veins. Optionally, the LV lead 204 may be inserted into the LV chamber or inserted along another vein or artery extending along the heart wall proximate to the LV. Optionally, the LV lead 204 may be formed as a patch or mesh net that is secured to or located adjacent to an exterior wall of the LV and/or the LA.

The LV electrodes 212-218 and/or RV electrodes 206-210 are utilized in various combinations to define different ATP excitation vectors. Examples of ATP vectors 220-228 are shown in FIG. 2A. For example, ATP vectors 220-223 extend between corresponding LV electrodes 212-218 and a common RV (coil) electrode 210. ATP vectors 224-226 extend between corresponding pairs of adjacent LV electrodes 212-218. ATP vectors 227 and 228 extend between corresponding pairs of non-adjacent spatially distributed LV electrodes (e.g., 218 and 214, and 218 and 212). The LV and RV leads 204 and 202 may be controlled to deliver unipolar or bipolar pulses separate from or as part of an ATP therapy and/or unipolar or bipolar prepulses before an ATP therapy.

Each LV and RV electrode 206-218 represents a potential sensing site and/or therapy site. When functioning as a sensing site, the corresponding LV and/or RV electrode 206-218 sense cardiac activity at the electrode position. The cardiac activity may represent intrinsic or paced normal sinus rhythms or intrinsic arrhythmic behavior. The cardiac activity may represent an evoked response which represents electrical activity that results following one or more stimuli that are induced at another site. Each stimulus (induced at one site) causes electrical activity to propagate along an activation pattern through at least a portion of the heart wall. The propagating electrical activity is sensed at RV and LV sensing sites as evoked responses.

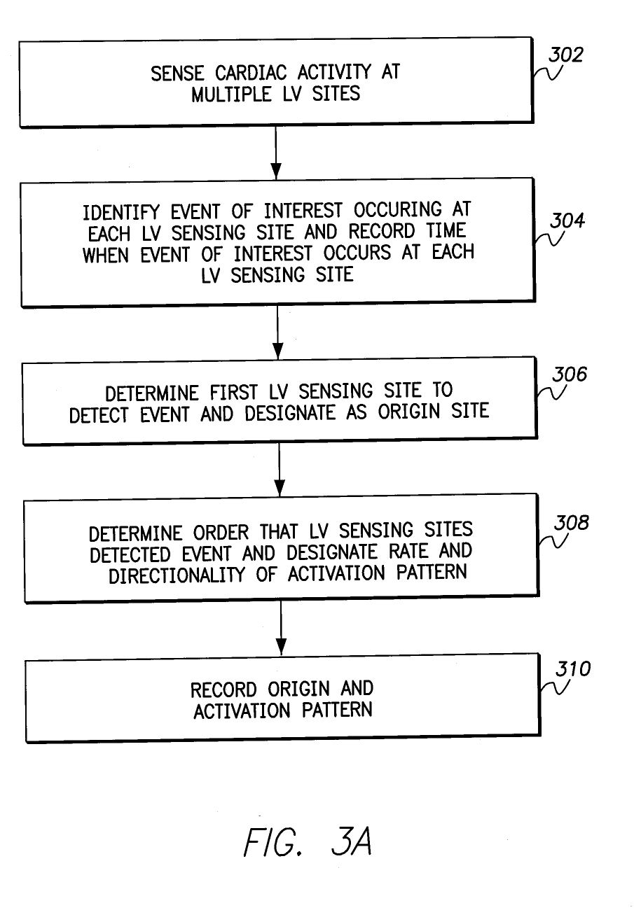

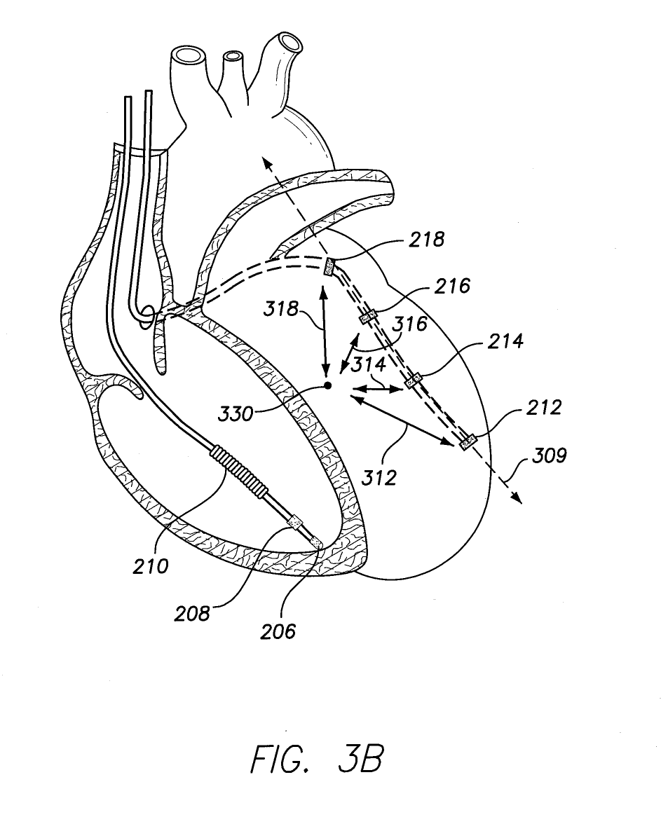

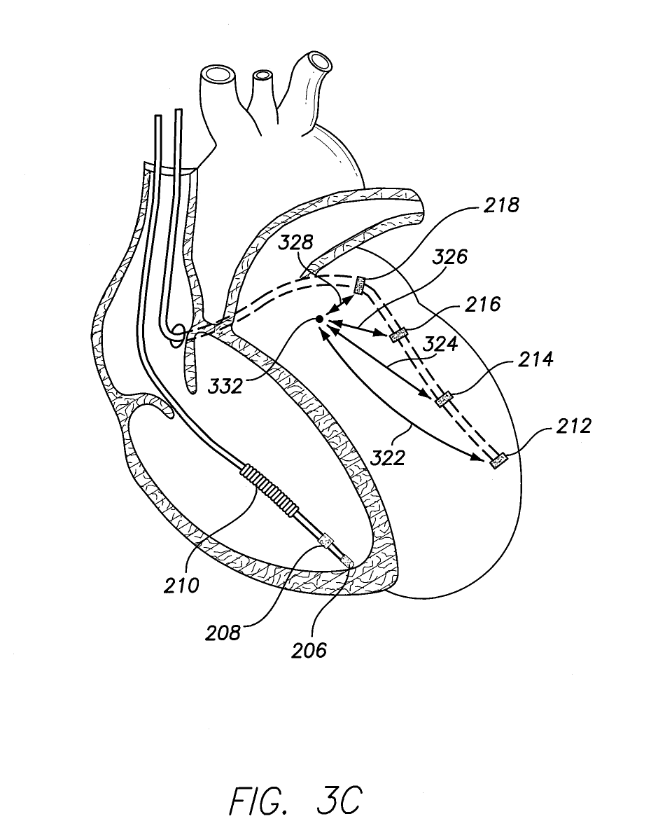

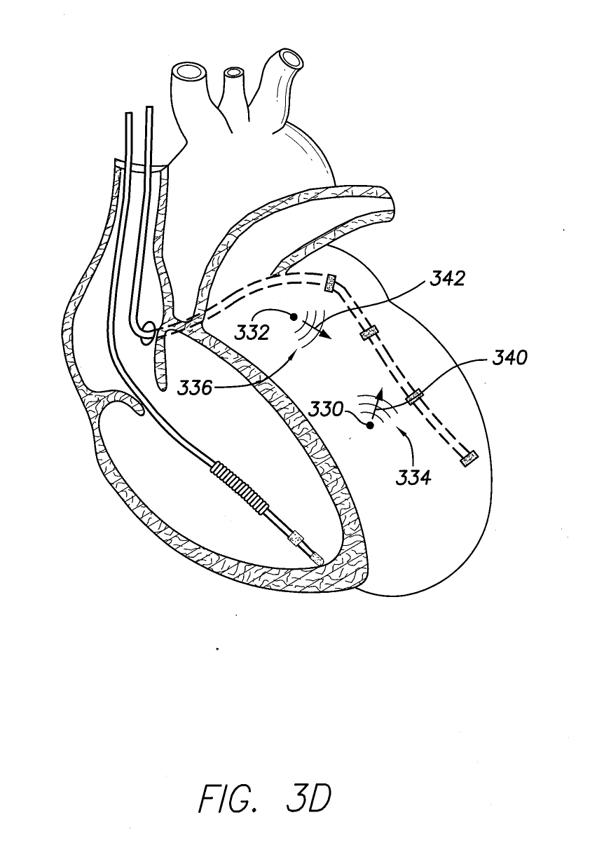

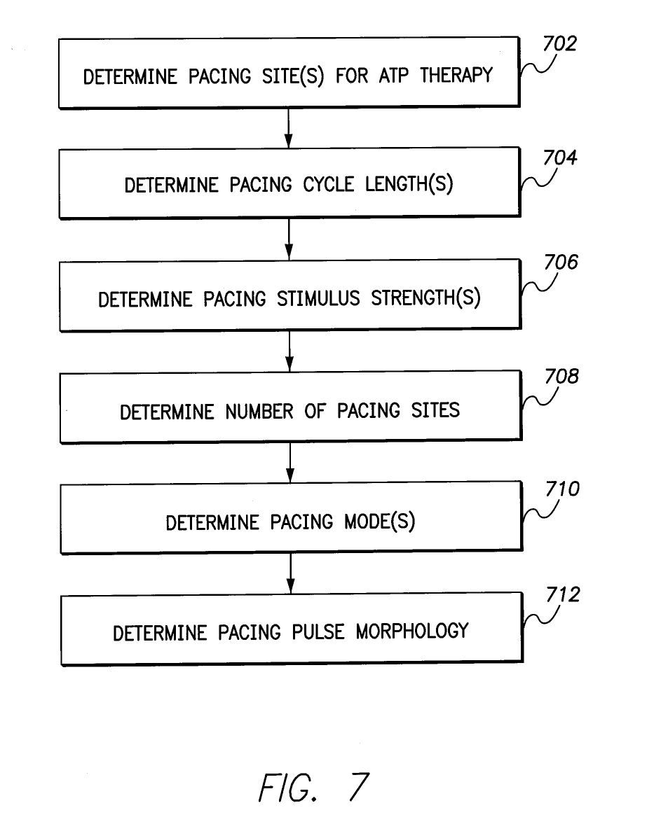

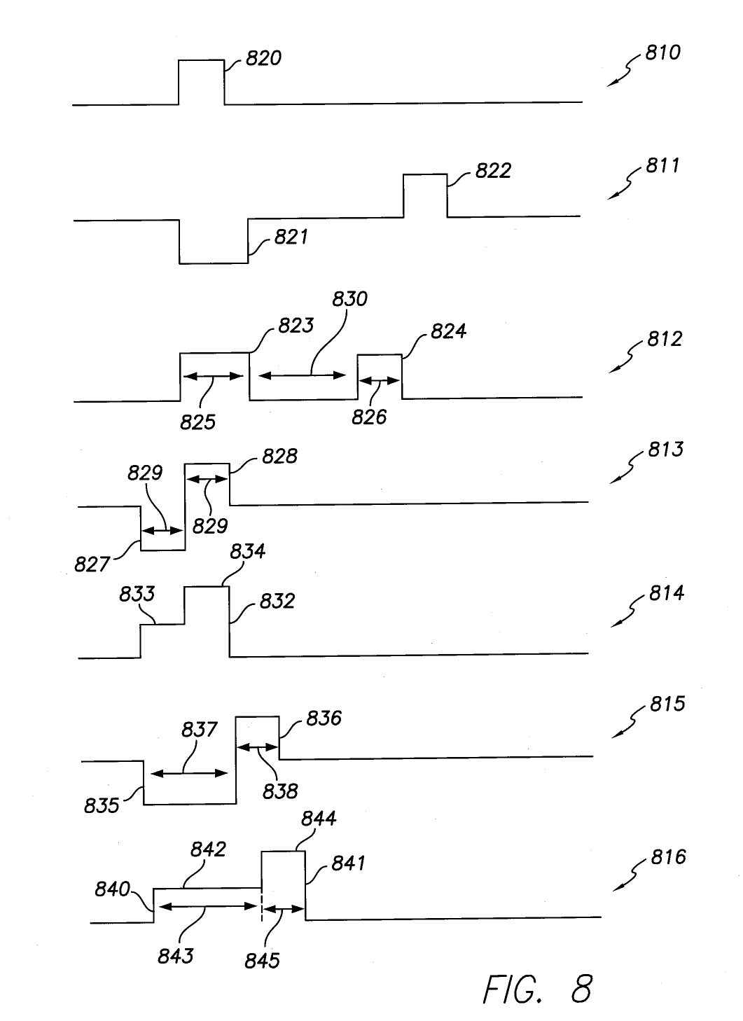

FIG. 2B illustrates a process carried out in accordance with an embodiment for delivering an ATP therapy. Beginning at 282, cardiac activity is sensed at one or more electrodes (e.g., LV, RV, RA and/or LA electrodes). At 284, the activation pattern is characterized, such as in accordance with one of the embodiments described herein (e.g., in connection with FIGS. 3-6). At 286, the method selects an ATP therapy and ATP parameters to be used during the ATP therapy. At 288, the method determines a pulse morphology. The operations at 286 and 288 may be carried out as explained below in connection with FIGS. 7 and 8 or in connection alternative parameter and morphology determination processes. At 290, the ATP therapy is delivered from one or more electrodes, such as from one or more LV electrodes.

At 292, the method performs an ATP capture confirmation process. The capture confirmation process determines whether the ATP therapy has been successful in capturing the heart muscle. By confirming capture, the method is able to determine whether the ATP parameters are effective or should be changed to improve efficiency and effectiveness. The sensing sites at which ATP capture is determined, is dependent in part on the therapy sites at which the ATP pulses are delivered.

FIG. 3A illustrates a flowchart for a method to characterize a VT activation pattern based on electrical activation sequence analysis in accordance with one embodiment. In the method of FIG. 3A, beginning at 302, cardiac activity is sensed at multiple LV sensing sites (e.g., 212-218). The cardiac activity may represent intrinsic or paced normal behavior or intrinsic abnormal behavior. A predetermined or programmed set or sets of the LV and RV electrodes are utilized for sensing based on a chosen type of analysis to characterize the VT activation pattern. Typically, monomorphic VTs (both focal and reentrant driven) exhibit a consistent ventricular activation pattern during VT. The multipolar LV lead 204 senses the electrical activity at multiple LV sites, providing the ability to characterize the VT activation pattern For example, a local VT activation pattern along the LV lateral wall may be characterized when the LV lead 204 includes multiple LV electrodes 212-218 along the LV lateral wall. In the method of FIG. 3A, an initial or earliest activation site and the sequence of activation is detected and used to identify directionality of a reentrant or focal activation during VT.

The signal at each sensing site is sensed as a waveform. The waveform includes certain common waveform segments and features (collectively “events of interest”). The events of interest at different sensing sites are detected temporally shifted from one another. As an example, an LV sensing site may detect a first local peak in the sensed signal at time T1, while an adjacent LV sensing site detects a similar second local peak in the corresponding sensing signal later, at time T2. The first and second local peaks were caused by the same reentrant or focal activation episode, and therefore represent common events of interest.

At 304, the sensed signals are analyzed to identify an event or events of interest. The event of interest may represent a point at which the sensed signal crosses a detection threshold. Alternatively, the event of interest may represent a peak or a local waveform segment that exceeds a baseline threshold. The analysis at 304 seeks to identify the common event of interest sensed at each LV sensing site and to identify the time at which the common event of interest was sensed at the corresponding sensing site.

FIGS. 3B and 3C illustrate examples of activation timings that may correspond to sensed events of interest. In the example of FIG. 3B, a focal episode 330 occurs which triggers an activation sequence. An event of interest from the activation sequence reaches and is detected by LV electrode 214 at time T1, while the same event of interest reaches and is detected by both LV electrodes 216 and 212 at time T2, and then is detected by LV electrode 218. By way of example, the time T2 may follow time T1 by 5 ms. Hence, the event of interest reaches LV electrode 214 first, and then reaches LV electrodes 212 and 216 at the same time, but 5 ms later, and then reaches LV electrodes 218 5 ms later.

In the example of FIG. 3C, a VT reentrant circuit begins at 332. When the reentrant activation at 332 occurs, an activation sequence is triggered and propagates across the heart, such as along the lateral wall of the LV. An event of interest from the activation sequence sequentially reaches the LV sensing sites. The event of interest may be detected by LV electrode 218 at time T4, while the same event is later successively detected by the LV electrode 216, then LV electrode 214, and then LV electrode 212 at times T5, T6 and T7, respectively. By way of example only, times T4 and T5 may be separated by 1 ms, while time T6 occurs 3 ms after time T4 and time T7 occurs 6 ms after time T4. At 304, the waveforms for the sensed signals from the multiple LV sensing sites are analyzed and the event(s) of interest are detected from each sensed signal. At 304, the method records the time(s) at which the event(s) of interest occurs at the corresponding LV sensing site. The recorded times represent the activation timing.

Next at 306 in FIG. 3A, the method determines the first LV sensing site to detect, in time, the event of interest and designates an origin site based thereon. In the above example of FIG. 3B, the first LV sensing site corresponds to LV electrode 214. In the example of FIG. 3C, the first LV sensing site corresponds to LV electrode 218. At 306, the first LV sensing site may be designated as the origin site (e.g., LV electrode 214 or 218 in the above examples). Hence, in one embodiment, the method designates the origin site to be the LV sensing site that first senses the event of interest.

Optionally, a more robust process may be utilized to position the origin site at, or proximate to, the focal ectopy 330, or at or proximate to the reentrant activation 332. The origin site may be designated as a point along the heart wall that is spaced apart from any of the LV electrodes 212-218. For example, when two or more LV electrodes sense the event at the same time (e.g., LV electrodes 212 and 216 in FIG. 3B), this may indicate that the origin of the event is located equal distance from the two LV electrodes and at a point transversely spaced from a reference line (e.g., line 309 in FIG. 3B) extending through the LV electrodes.

With reference to FIG. 3B, to locate the origin site at or proximate to ectopy 330, the times T1-T4 may be used to calculate radial distances 312-318 from the LV electrodes 212-218. The radial distances 312-318 may be calculated based on a known spacing between the LV electrodes 212-218 and the relation between recorded times T1-T4 at which each LV electrode 212-218 sensed a common event. Optionally, a rate of electrical propagation may be utilized in the calculation of radial distances 312-318. The rate of propagation represents a rate at which electrical activity moves in a given direction through the heart wall. The rate of propagation may be pre-assigned, programmed, measured periodically, measured during an arrhythmia episode or determined otherwise. The radial distances 312-318 define circles or arcs surrounding each LV electrode 212-218. The origin site may be designated as the point of intersection between the circles, defined by the radial distances 312-318.

In FIG. 3B, the times T1-T4 are used to calculate the radial distances 312-318 that define the circles which intersect to locate the ectopy 330. The origin site is designated to be proximate to ectopy 330. In FIG. 3C, the times T5-T8 are used to calculate the radial distances 322-328 that define the intersecting circles that locate the ectopy 332 and designate the origin site to be proximate to ectopy 332.

Returning to FIG. 3A, at 308, the method determines the sensing order in which the LV sensing sites detect the common event(s) of interest. The sensing order is then used to calculate a directionality of an activity pattern or sequence that propogates from the ectopy. Optionally, at 308, the method may also calculate the rate at which the activation pattern or sequence propagates from the ectopy.

FIG. 3D illustrates examples of different activation patterns 334, 336 associated with different ectopys. Each activity pattern 334, 336 has an origin of the episode 330, 332 and a direction 340, 342 along which the electrical activity associated with the ectopy 330, 332 propogates. Each activity pattern 334, 336 also has a rate of propagation, at which the electrical activity moves along the wall of the heart.

At 310, the origin and activation pattern are stored for later analysis and use, such as in connection with determining an ATP therapy. The first target site for delivery of an ATP therapy may then be at or near the initial or earliest activation site. For example, the ATP therapy site may be set to correspond to the LV sensing site that was first to detect the activation sequence.

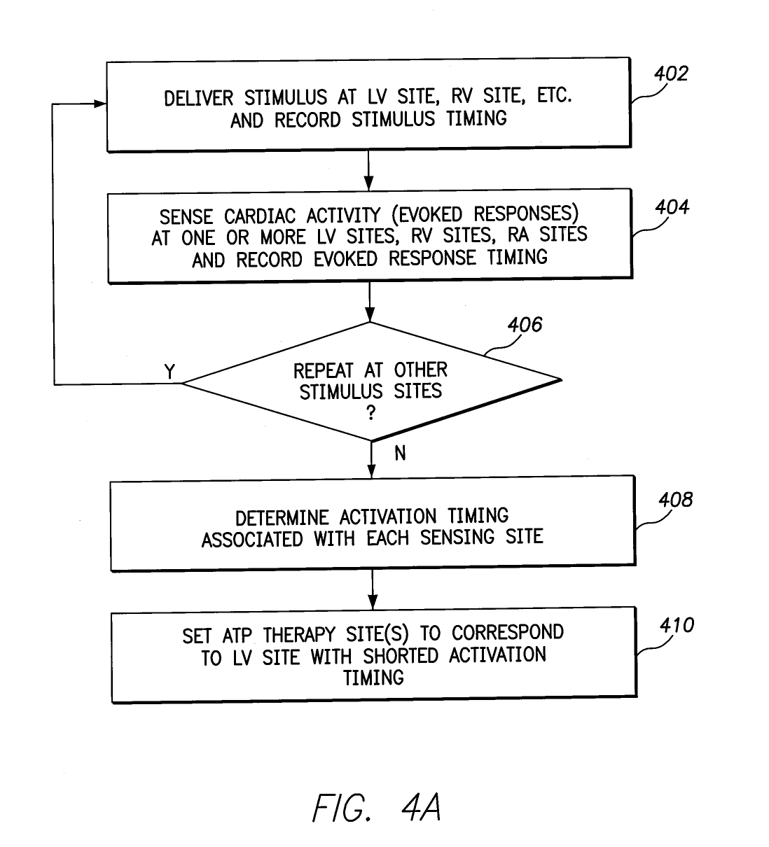

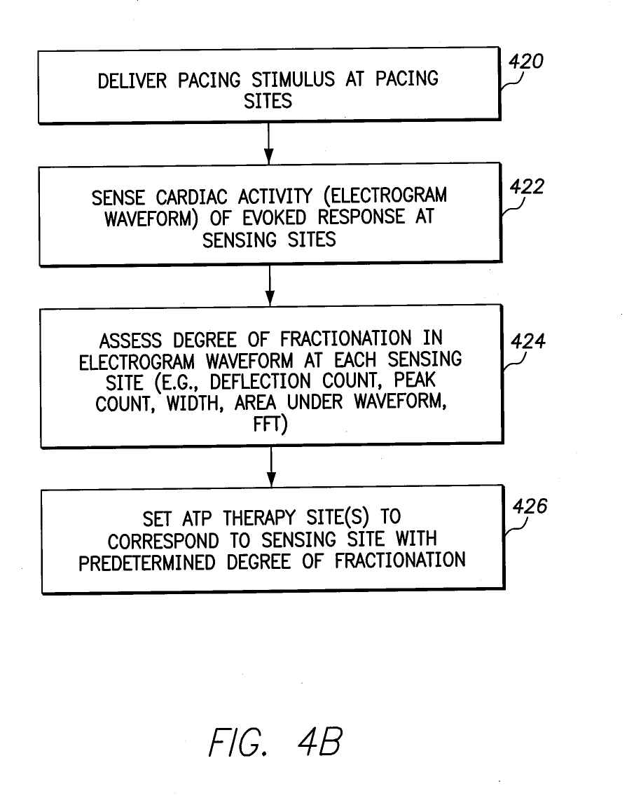

FIG. 4A illustrates a flowchart for a method that utilizes VT capture analysis to characterize VT activation patterns in accordance with an alternative embodiment. At 402, a pacing pulse is delivered during VT at one or more electrodes at LV sites, RV sites, RA sites and/or LA sites. For example, the stimulus may be delivered at one or more LV electrodes 212-218 and at one or more of RV electrodes 206-210. The stimulus may be delivered along one or more of the vectors 220-228 shown in FIG. 2. The stimulus triggers a stimulus induced activation pattern that propagates across at least a portion of the heart. An event or events of interest from the stimulus induced activation pattern propagates as an evoked response(s) across the heart. The evoked response(s) are sequentially detected at each LV sensing site.

At 404, the evoked responses are sensed at all or a set of the LV sensing sites (e.g., at 212-218). Optionally, the evoked responses may be detected in the far field at RA electrodes. Optionally, the evoked responses may be sensed at all or a portion of the RV sensing sites, RA sensing sites and/or LA sensing sites (if available). The method records the times for when each sensing site detects the corresponding evoked response. The recorded times may be at common or different points in time depending upon a location of the pacing pulse relative to the sensing sites, to the direction of propagation and to the rate of propagation. At 406, it is determined whether to repeat the operations at 402 and 404 for stimuli delivered at one or more other LV electrodes or along one or more other vectors.

For example, during the operations at 402-406, a pacing pulse or, a first train of stimuli pulses, may be delivered at LV electrode 214 and the evoked responses detected at LV electrodes 208 and 210. Then a second pacing pulse or train of pulses may be delivered at LV electrode 218 and the evoked response detected at LV and RV electrodes 208-216. Optionally, the evoked responses may be detected in the far field at RA electrodes. By way of example, the train of pulses may be delivered at a rate corresponding to a predetermined percentage (e.g., 80-95%) of the VT cycle length. Once it is determined that all desired pulses are delivered and all desired evoked responses are sensed, flow moves from 406 to 408.

At 408, the activation timing of the evoked responses is determined for the plurality of sensing sites. The activation timing for an evoked response at a given sensing site represents a time period between an end of a pacing pulse (or the end of the train of pulses) and a time at which the associated evoked response is sensed at the corresponding sensing site. For example, following a pacing pulse or train of pulses at LV electrode 218, activation times may be measured at the LV electrodes 212 and 214, and the RV electrodes 206-210. One or more separate pulses may be delivered at the LV electrodes 216 and 214 and 212, for each of which activation times are determined relative to the other LV electrodes 212-218 and the RV electrodes 206-210. By way of example, the activation timing associated with a sensing site may be 5 ms, 10 ms and the like. In general, some sensing sties will have short activation timing while other sensing sites will have long activation timing.

At 410, an initial ATP therapy site is determined. For example, the initial ATP therapy is determined to be the LV sensing site that satisfies an activation time threshold (e.g., having the shorted activation time relative to the other LV sensing sites). For example, the activation times may be identified at 408, to be 3 ms between LV electrodes 218 and 216, 6 ms between LV electrodes 216 and 214, and 7 ms between LV electrodes 214 and 212. Hence, LV electrode 218 may be designated as the initial ATP therapy site. In the above manner, capture testing and analysis of evoked responses is utilized to identify an initial site at which ATP therapy is to be delivered.

FIG. 4B illustrates a flow chart for a method that utilizes wave morphology analysis to characterize VT activation patterns in accordance with an alternative embodiment. At 420, a pacing pulse is delivered at one or more electrodes. At 422, electrogram waveforms for the evoked responses from the patient stimulus are sensed at predetermined sensing sites. For example, the sensing sites may include LV electrodes 212-218 and/or RV electrodes 206-210, or a subset or combination thereof.

At 424, the method assesses the degree of fractionation within the electrogram waveform associated with each sensing site. The degree of fractionation within an electrogram is representative of an amount of wave-front collision that occurs within a region. The degree of fractionate within the electrogram is indicative of whether a region within the heart exhibits slower conduction or blocks conduction. As an activation sequence propagates from the paced stimulus, the activation sequence encounters regions of the heart that have different rates of conduction or may block conduction entirely. In these regions where conduction is blocked or slowed down, discontinuities are created within the wave-front of the activation sequence. These discontinuities in the wave-front subsequently collide with one another. The wave-front collisions and conduction blocking/degradation are exhibited in the electrogram by way of a fractionation or excessive change in the waveform of the electrogram.

The degree of fractionation within the electrogram can be assessed based on various parameters of the waveform. Examples of these parameters include counting the number of deflections within a waveform and choosing the waveform with the most deflections as the most fractionated waveform, counting the number of peaks within a waveform and choosing the waveform with the most deflections as the most fractionated waveform, determining a width of a waveform segment of interest and choosing the waveform with lowest width as the most fractionated, and the like. Further examples of the parameters that may be assessed include calculating the area under a waveform segment (e.g., such as determining the integral of the waveform) or performing a Fast Fourier Transform (FFT) of the waveform and analyzing the outcome of the FFT. The degree of fractionation may differ at different sensing sites. At 424, the method determines some relative relation between the amount of fractionation within the electrograms at various desired sensing sites.

At 426, the method sets an initial ATP therapy site to correspond to the sensing site or sensing sites that have a predetermined degree of fractionation. For example, the initial ATP therapy site may be set to the sensing site having the least degree of fractionation as determined through testing in at a time prior to the current VT. Alternatively, the initial ATP therapy site may be set to a pair or a subset of sensing sites having a degree of fractionation below a fractionation threshold.

In the example of FIG. 4B discussed above, the process is described in connection with delivery of one or more pacing pulses. Optionally, the process of FIG. 4B may be repeated with respect to multiple pacing pulses from multiple pacing sites. For example, the operations at 420 and 422 may be repeated for multiple pacing sites in order that electrogram waveforms for evoked responses are sensed at a plurality of sensing sites in connection with different pacing sites. At 424, the assessment of the degree of fractionation may be performed not only for multiple sensing sites but for each of the desired sensing sites relative to multiple pacing sites. By increasing the number of pacing sites and the number of sensing sites, the assessment at 424 may become more robust and better identify a preferred initial ATP therapy site and ATP therapy configuration.

Optionally, in accordance with an alternative embodiment, electrode impedance analysis may be utilized to characterize VT activation patterns. When utilizing electrode impedance, the electrode impedance is measured at multiple sensing sites during sinus rhythm or during ventricular pacing. The electrode impedances are then analyzed to identify the sensing site having a predetermined desired impedance characteristic (e.g., for example a lowest level of impedance or a highest level of impedance). Thereafter, the initial ATP therapy site may be set to be the sensing site having the desired level of electrode impedance, such as the sensing site with the minimal electrode impedance. By way of example, the impedance measurement may be in connection with determining a pacing threshold associated with an electrode or a vector. The initial ATP therapy site may be set to be the sensing site having the lowest pacing threshold.

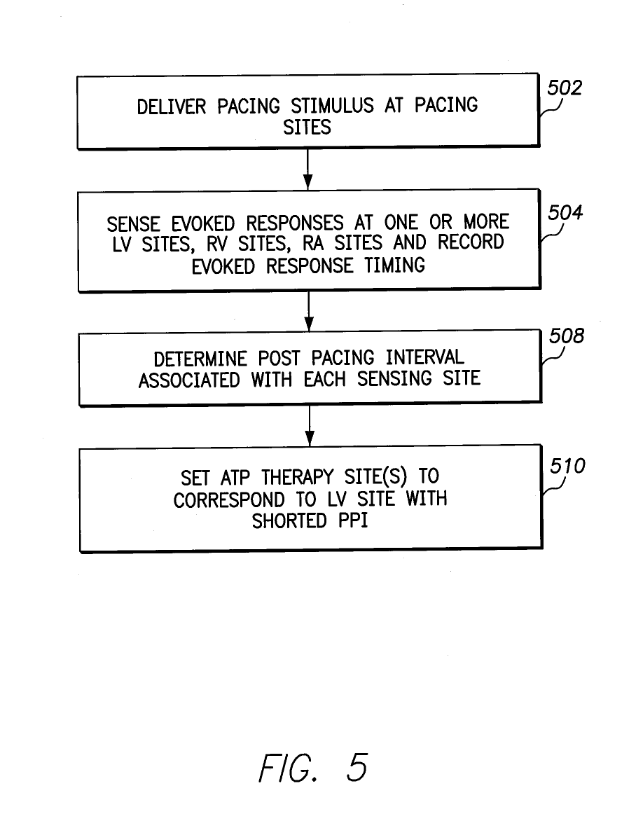

FIG. 5 illustrates a flowchart for a method that utilizes post PPI analysis to characterize VT activation patterns in accordance with an alternative embodiment. At 502, one or more pacing pulses is delivered at one or more pacing sites. For example, the pacing pulsing may be delivered in the RA from a pacing electrode (e.g., as shown in FIG. 1). Optionally, the pacing pulses may be delivered at one or more of LV electrodes 212-218 and at one or more of RV electrodes 206-210. The pacing pulse triggers a paced activation sequence that propagates across at least a portion of the heart. For example, the pacing rate may be at a rate slightly faster than a tachycardia cycle length. At 504, the method senses, at one or more sensing sites, post-pacing local intrinsic activation. Optionally, the sensing sites may be at all or a portion of the LV sensing sites, RV sensing sites, RA sensing sites and/or LA sensing sites (if available). The post-pacing local intrinsic activation follows the last paced stimulus by some measurable period of time, referred to as the PPI. At 504, the PPI times are also recorded for when sensing sites detect the corresponding evoked responses. The PPI times may be at a common or different point in time. The PPI times depend upon a location of the pacing pulse relative to the sensing sites, depend on the direction of propagation and depend on the rate of propagation.

This interval is an indication of the proximity of the pacing site to the reentry circuit and is the time from the pulse to the next non-stimulated depolarization. At sites in the circuit, the orthodromic wavefront returns to the pacing site after one revolution through the reentry circuit, so the PPI from the last pacing pulse is equal to the revolution time through the circuit which is equal to the tachycardia cycle length plus the return time to the pacing or sensing site(s).

At sites outside of the reentrant circuit, the PPI is longer because the pacing site is remote from the reentrant circuit and the pacing pulse must travel to the reentrant circuit, through the reentrant circuit, and then back to the pacing site. The PPI may also exceed the tachycardia cycle length when there is decremental conduction within the reentrant circuit. For example, the PPI for ischemic VT may be within 30 ms of the tachycardia cycle length.

At 508, activation timing of the PPI is measured at a plurality of sensing sites. For example, following a pulse train at LV electrode 218, the PPI may be measured at the LV electrodes 212 and 214, and the RV electrodes 206-210. Separate pacing pulses or pulse trains may be delivered at the LV electrodes 216 and 214 and 212, for each of which the PPI are determined relative to the other LV electrodes 212-218 and relative to the RV electrodes 206-210. By way of example, the PPI associated with a sensing site may be 5 ms, 10 ms and the like following the last pacing pulse. In general, some sensing sites will have short PPIs while other sensing sites will have long PPIs.

At 510, the initial ATP therapy site is determined to be the LV sensing site having the shortest PPI relative to the other LV sensing sites. For example, the PPI may be identified at 508, to be 10 ms for LV electrode 218, 20 ms for LV electrode 216, and 30 ms for LV electrode 214. Hence, LV electrode 218 may be designated as the initial ATP therapy site. In the above manner, PPI testing and analysis is utilized to identify an initial site at which ATP therapy is to be delivered.

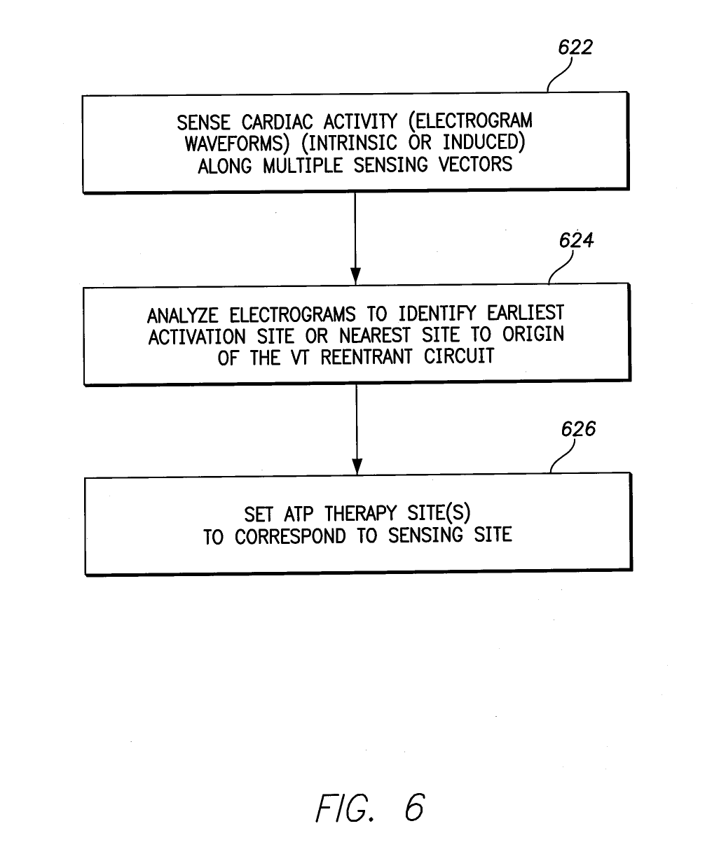

FIG. 6 illustrates a flow chart for a method that utilizes electrograms to characterize VT activation patterns in accordance with an alternative embodiment. Beginning at 622, the method senses electrogram waveforms along multiple sensing vectors. The sensed waveforms may be due to intrinsic rhythmic or arrhythmic behavior. Alternatively, the waveforms may be responsive to induced behavior (such as responsive to a paced pulse, an ATP stimulus or otherwise). The waveform sensed at 622 may be responsive to paced pulses delivered from a pacing lead, from an RA lead, an LV lead and/or an RV lead. Examples of sensing vectors along which electrograms may be acquired include 1) the RV tip to RV ring/coil, 2) LV tip to ring; 3) LV tip to RV ring/coil; 4) RV tip to can; 5) LV tip to can; 6) LV ring to can; 7) any electrode (RA, RV or multi LV) to SVC coil (if available); 8) SVC or RV coil to any other electrode (RA, RV and multi-LV); and 9) any ventricular electrode (RV or multi-LV) to RA tip or ring.

At 624, the electrograms are analyzed to identify a characteristic, such as the activation site or nearest site to the origin of a VT reentrant circuit. By analyzing multiple vectors that include an LV electrode, the method of FIG. 6 provides improved characterization of the VT activation pattern. For example, the electrograms that are acquired from sensing vectors that include LV electrodes 212-218 (FIG. 2), may be used to better identify an origin of a focal VT ectopy or an origin of a reentrant VT circuit.

At 626, an initial ATP therapy site is set to correspond to one or more sensing sites based on the analysis performed at 624. For example, the initial ATP sensing site may be set to correspond to the earliest activation site. Alternatively, the initial ATP therapy site may be set to correspond to the origin of a VT reentrant circuit. As a further option, the initial ATP therapy site may be set to correspond to a sensing site that is estimated to be closest (relative to other sensing sites) to the VT focal origin or the VT reentrant circuit origin.

FIG. 7 illustrates a flow chart of a method that selects parameters for the ATP therapy configuration in accordance with an embodiment. The parameters for an ATP therapy may be programmed to various levels, such as between 0 and 100 percent relative to an aggressiveness standard. An aggressiveness of an ATP therapy is represented by a combination of various ATP parameters, such as the pacing rate, pacing amplitude, S1, S2 intervals, S1, S2, S3 intervals, the number of pacing pulses (S1), the number of S2/S3 pulses, the burst for ramp shape of an ATP therapy, multi-site LV ATP or single site LV ATP, sequential or simultaneous LV pacing and the like. The ATP configuration may be changed by changing ATP parameters such as pacing sites, pacing cycle length (e.g., burst length, ramp length, etc.), the number of pacing pulses, pacing vectors and/or pacing amplitude.

Beginning at 702, the method determines a pacing site or pacing sites to be used during delivery of an ATP therapy. As one example, an initial pacing site may be used during an initial stimulus train of the ATP therapy, followed by delivery of an ATP stimulus train at a different pacing site or pacing sites. As a further example, an initial portion of a train of ATP stimulus pulses may be delivered at one pacing site, followed by separate trains of pacing pulses delivered at one or more additional different pacing sites. The location and combination for the initial and each subsequent pacing operation may be based on one or more of the VT characterization processes described herein (such as the processes described in connection with FIGS. 2-6).

At 704, the method determines the pacing cycle length or pacing cycle lengths to be utilized in the ATP therapy configuration. For example, the pacing cycle length may be initially set at a percentage of the VT cycle length (e.g., 95%). Thereafter, during each subsequent ATP therapy cycle, the pacing cycle length may be changed (e.g., decremented by one or more percent). This iterative process of decreasing the pacing cycle length may continue until the ATP therapy is determined to lose capture or to terminate the VT ectopy. Optionally, the initial pacing cycle length percentage may be programmed by a physician. Optionally, the amount may be programmed by a physician for which the pacing cycle length is decremented during each iterative ATP therapy.

At 706, the method determines the pacing pulse strength to be delivered during each pacing pulse of the ATP therapy. Optionally, more than one pacing pulse strength (e.g., voltage, pulse width, etc.) may be utilized during a single ATP therapy. For example, during an ATP therapy, a train of pacing pulses may be delivered wherein a first series of the pacing pulses have a first strength (voltage, width, etc.), while a second series of the pacing pulses have a greater or lesser second strength.

When a single site or multi-site ATP therapy configuration is selected, an initial attempt is delivered with a predetermined number of ATP pulses. When the initial series of ATP pulses fails to terminate the VT episodes, the output stimulus voltage may be increased for a subsequent ATP therapy. Optionally, in addition to, or in place of, increasing the stimulus voltage during subsequent ATP pulses, the pulse width of each ATP pulse may be increased during one or more of the later ATP pulse trains. It may be desirable with bi-polar pace configurations to increase the stimulus voltage and/or pulse width at successive ATP pulse trains where it is possible to achieve both cathodal and anodal capture at higher energy outputs. During unipolar modes, higher stimulus strengths will also achieve good tissue penetration and capture a large portion of the tissue, particularly the tissue of importance within transmural VT circuits.

At 708, the method determines the number of pacing sites to be utilized during an ATP therapy. For example, a single LV electrode site may be used during ATP therapy to deliver pulses. Alternatively, multiple LV electrode sites may be utilized to deliver pacing pulses during ATP therapies. Optionally, any number of LV electrodes may be utilized to deliver ATP therapies when the timing of the sensed activation patterns in the left ventricle indicates that a heterogenous pattern of activation exists. The method may determine to utilize multi-site LV based ATP during a first attempt or initial ATP therapy. This multi-site LV based ATP therapy may be utilized before any single site based ATP therapy is delivered. The multi-LV based ATP therapy may use only LV electrodes to deliver the ATP therapy. Alternatively, the multi-site LV based ATP therapy may utilize both LV electrodes and RV electrodes to perform bi-ventricular ATP pacing. When multi-site LV based ATP therapy is chosen, the LV electrodes may be set in a bipolar configuration or combination that utilizes the RV ring as an anode. Optionally, another RV electrode may be utilized during bipolar configurations with LV electrodes. It may be preferable to utilize a bipolar configuration (with or without an RV ring as an anode) to afford anodal capture on the return electrode in order to provide a relatively large captured area within the ventricles.

At 710, the method of FIG. 7 determines the pacing mode or pacing modes to be utilized during the ATP therapy. For example, the pacing mode may include simultaneous or sequential ATP pacing. When utilizing multi-site LV pacing, the method may determine to use simultaneous multi-site LV ATP therapy or sequential multi-site LV ATP therapy. When sequential multi-site ATP therapy is selected, a sequence of pacing pulses and the inter-LV pacing timing may be selected to follow the VT activation pattern.

For example, the sequence of electrical activation recorded from four LV electrodes during a VT episode may be electrode 218, followed by electrode 216, followed by electrode 214, followed by electrode 212. The multi-site LV sequential pacing ATP therapy may be delivered in the same sequence, namely ATP pulses may be delivered from electrode 218, then from electrode 216, then from electrode 214, and finally from electrode 212. The inter-LV pulse timing may be programmed or otherwise selected (e.g., between 4 and 100 ms). As a further option, the inter-LV pulse timing may be set to correspond to the difference in the activation time between the LV electrodes where the activation timing is recorded during a VT episode. For example, when an activation time of time T70 is detected between electrodes 218 and 216, activation time T72 is recorded between electrodes 216 and 214, and activation time of T74 is recorded between electrodes 214 and 212. These activation times T70, T72 and T74 may be used as the inter-LV pulse timing during delivery of sequential ATP pulse trains from each of the electrodes 218-212. Alternatively, the shortest of the activation times T70-74 may be chosen to be the inter-LV pulse timing between all multi-site LV ATP pacing trains.

In certain instances, the timing of the sensed activations at the LV electrodes may indicate a relatively heterogenous pattern of activation. When a heterogenous activation pattern is detected, the combination of sequential and/or simultaneous ATP pulse trains may be delivered such that the earliest site of activation is targeted by an ATP pulse train from a single LV electrode. Once the initial ATP therapy site (e.g., LV electrode) has delivered an initial ATP therapy train, next simultaneous ATP therapies are delivered from the remaining LV therapy sites. As another example, a first ATP therapy may be delivered simultaneously from a pair of LV electrodes (e.g., LV electrodes 218 and 216), followed sequentially by an ATP therapy delivered simultaneously from a second pair of electrodes (e.g., electrodes 214 and 212).

The parameters for pacing sites, pacing cycle length, pacing pulse strength, number of pacing sites and pacing modes determined at 702-710 may be based on one or more of the analyses performed in connection with the methods of FIGS. 2-6. As a further option, the analysis for VT characterization from the methods of FIGS. 2-6 may be combined such that two or more of the analysis techniques are utilized and the results aggregated through a weighting process or otherwise to then select the ATP parameters for the ATP therapy.

As one example, ATP options may include an RV ATP therapy, BV ATP therapy, LV ATP therapy from a single LV electrode, multi-site LV ATP therapy and multi-site LV BV ATP therapy. During multi-site LV BV ATP therapy, ATP pulses are delivered from multiple LV electrodes during the BV therapy. As one example, a suggested sequence may be to first deliver an RV ATP pulse train, followed by a BV pulse train, followed by a multi-site LV ATP pulse train, followed by a multi-site LV BV ATP pulse train. Optionally, only a subset of the foregoing exemplary ATP pulse trains may be delivered or different combinations of the foregoing ATP pulse trains may be delivered. The electrodes utilized and the parameters of the ATP therapy may be varied depending upon the activation pattern of the VT episode and/or the location of a reentrant circuit.

Returning to FIG. 7, at 712, the method determines a pacing pulse morphology. At 712, in FIG. 7, the pacing pulse morphology is determined in an effort to increase a likelihood of capture. To lower the capture threshold for ATP therapy or increase the likelihood of capture, a depolarizing or hyper-polarizing pre-pulse may be used. The pre-pulse based ATP therapy may create a wide, uniform and faster wavefront propagation that may be effective to obtain the excitable gap desired for VT entrainment. Optionally, monophasic and biphasic ATP therapies may be utilized to achieve the benefits of a pre-pulse based ATP therapy. At 712, the pacing pulse morphology includes the determination of parameters for monophasic, biphasic or pre-pulsed shapes such as the hyperpolarizing first pulse and depolarizing second pulse shapes; 2) the depolarizing first pulse shape and hyperpolarizing second pulse shape; 3) the duration of each pulse; 4) the timing of each pulse; 5) the amplitude of each pulse; 6) the number of depolarizing pulses; and 7) the number of hyperpolarizing pulses. The example of FIG. 8 illustrates only a few of the potential options that may be chosen for the pulse parameters for the monophasic, biphasic or prepulsed morphologies.

FIG. 8 illustrates examples of alternative pre-pulse and/or pacing pulse morphologies 810-816. For example, pacing pulse morphology 810 may include a single positive pulse 820. Pacing pulse morphology 811 may include an initial negative pacing pulse 821 followed by a positive pacing pulse 822. Pacing pulse morphology 812 may include positive pacing pulses 823 and 824 which differ in pacing pulse width 825 and 826. Pacing pulse morphology 813 may include successive negative and positive pulses 827 and 828 of equal width 829. The pacing pulses 827 and 828 occur successively without any delay therebetween. The pacing pulses 823 and 824 are separated by a delay 830.

The pacing pulse morphology 814 includes a single pulse 832 having a stepped positive amplitude with first and second steps 833 and 834. The pacing pulse morphology 815 includes successive negative and positive pacing pulses 835 and 836 without any delay there between, where the negative pacing pulse 835 has a longer duration 837 than the duration 838 of positive pulse 836. The pacing pulse morphology 816 includes a series of positive pacing pulses 840 and 841 that are joined to one another without any delay therebetween. The pacing pulse 840 has a first amplitude and pulse width 842 and 843, respectively. The amplitude 842 is less than the amplitude 844 of the subsequent pulse 841. The pulse width 843 of the first pulse 840 is longer than the pulse width 845 of the second pulse 841. Optionally, alternative pulse widths and amplitudes may be utilized. Similarly, alternative pulse intervals and delays between pulses as well as the number of pulses may be varied accordingly.

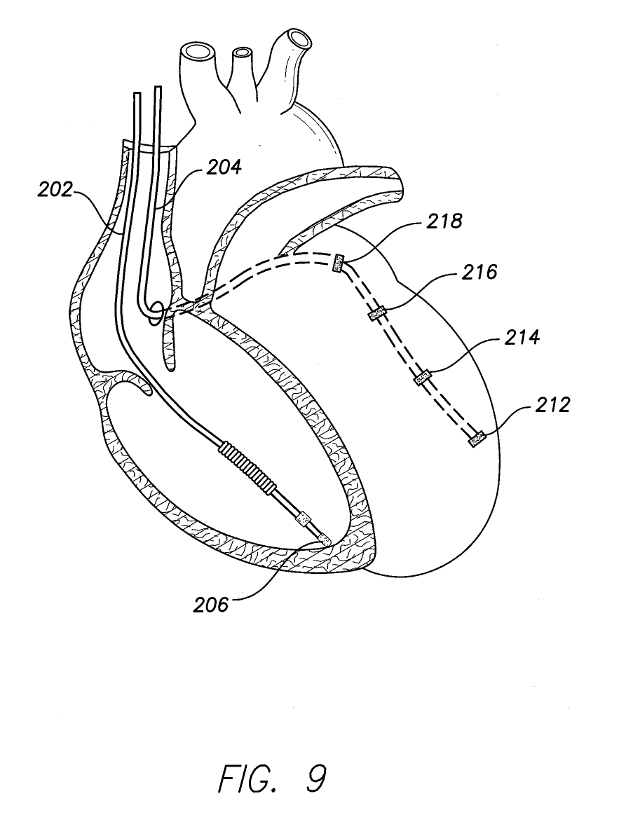

FIG. 9 illustrates a graphical example of the manner in which ATP therapy electrodes and ATP captured confirmation may be performed in accordance with an embodiment. In FIG. 9, the RV electrode 202 is utilized to deliver the ATP therapy. For example, the RV tip electrode 206 may be utilized to deliver ATP pulses. When the ATP therapy is delivered in the right ventricle, the LV electrode 204 is configured to perform the ATP capture confirmation. To perform ATP capture confirmation, one or more of the LV electrodes 212-218 are set to sense cardiac activity following the ATP pulse train delivered at electrode 206. When the LV electrodes 212-218 detect capture based on the ATP pulse delivered in the right ventricle, this may be an indication that the ATP therapy is effective. When capture is not detected, the ATP therapy may be adjusted, such as to change the number of pacing pulses, the pacing pulse strength, the site at which the ATP pulses are delivered and the like. The ATP therapy may continue to be adjusted until capture of a reentrant circuit is achieved utilizing a predetermined number of pacing pulses (e.g., a minimum number or number of pacing pulses below a threshold). It is desirable in certain instances to limit the number of pacing pulses needed for ATP therapy in order to similarly limit any likelihood that pro-arrhythmic affects may arise due to the ATP pacing. The ATP capture confirmation process at 292 may be generally performed in connection with intra-chamber ATP therapy, namely when the ATP therapy is delivered only from one chamber of the heart. For example, when the ATP therapy is delivered only from the RV lead 202, the LV lead 204 may be utilized for capture confirmation. Alternatively, the RV lead 202 may be utilized to sense capture and to confirm whether the ATP therapy is effective.

Optionally, the single chamber ATP capture confirmation process described above in connection with FIG. 10 and the operation at 292 in FIG. 9 may be performed prior to performing an inter-chamber ATP therapy. In this example, ATP may be delivered from one or more ventricular electrodes. Following the delivery of the pulse from one or more ventricular electrodes used for ATP, a unipolar EGM may be taken from non-paced ventricular electrode(s). In this unipolar electrogram, a negative depolarization of a particular electrogram would be indicative of capture for that particular electrode. Further, a biphasic unipolar electrogram with a first positive deflection and a second negative deflection recorded from an electrode neighboring that at which the ATP stimulus was delivered indicates both capture and exit from the ATP pacing electrode. The timing from the ATP pulse to the activation time recorded at non-paced electrodes will be measured. In order to be qualified as capture during VT, the sensed electrograms will exhibit one or more from the following: 1) activation time (i.e., ATP pulse to sensed activation) being shorter than the VT cycle length; 2) changes in morphology; 3) changes in regularity; and 4) VT termination. As a further option, the capture confirmation may be performed utilizing hemodynamic sensors which obtain hemodynamic sensor data. Hemodynamics may be measured prior to and after initiation of the ATP, and compared when there is further deterioration in the hemodynamics after initiation of the ATP. Then the ATP therapy may be determined to not achieve capture or to be ineffective. Hence, hemodynamics or single chamber ATP capture confirmation may be utilized in connection with determining whether to change the parameters or settings of the ATP therapy.

When an ATP therapy is delivered, in certain instances, the therapy may result in acceleration of an otherwise regular tachycardia to an irregular tachycardia. As a further example, in certain instances, an ATP therapy may convert a monomorphic VT into a polymorphic VT. The potential for an ATP therapy to convert tachycardias from regular to irregular or VTs from monomorphic to polymorphic is dependent in part upon whether the ATP parameters are set to desired levels. For example, when the pacing cycle length is set too short (too fast), this may limit the effectiveness on a tachycardia or VT. The ATP capture confirmation can be performed to obtain electrograms from an opposite chamber of the heart. The electrograms from the opposite chamber can be analyzed for regularity or irregularity or the degree of fractionation as explained above. The regularity of an electrogram may be determined based on the R-to-R interval, a fast fourier transform analysis of the electrogram, a morphologic analysis of the electrogram, or an integration analysis of the area under the electrogram and the like. Optionally, far field ventricular activation from an RA electrode may also be analyzed in connection with determining ventricular regularity during an ATP therapy. When irregularity in the ventricular activation is detected, an ATP therapy may be immediately stopped and the settings for the parameters of the ATP therapy adjusted.

In accordance with embodiments described herein, ATP therapies are delivered to terminate VT. The ATP therapies are delivered to capture/entrain a reentrant circuit and interrupt reentrant activation. Factors that are involved in the effectiveness of an ATP therapy include the location at which the therapy is delivered, the pacing cycle length and the number of pacing pulses, among other things. Multi-site LV ATP will increase a likelihood of capture/entrainment of the reentrant circuit by capturing a larger area and creating more uniform and faster activation propagation across the heart wall. By increasing the capture area and creating a more uniform or faster activation sequence, embodiments are particularly beneficial when a location of a VT reentrant circuit is unknown.

In accordance with embodiments, ATP therapies are described that terminate VF. To terminate VF, it is desirable to obtain a large capture area sufficient to reach a critical mass of the fibrillating myocardium. ATP pacing vectors are selected such that anodal pacing can be achieved in addition to cathodal pacing. For example, LV bipolar configurations with high output or LV electrode to RV ring or coil configurations with high output may be desirable pacing vectors utilized to capture the desired critical mass of a fibrillating myocardium.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. An implantable medical device, comprising:
a lead configured to be located proximate to the left ventricle (LV) of the heart, the lead including multiple LV electrodes to sense cardiac activity at multiple LV sensing sites;
a detection module to detect an arrhythmia that represents at least one of a tachycardia and fibrillation based at least in part on the cardiac activity sensed at the multiple LV sensing sites; and
an ATP therapy module to identify at least one of an ATP configuration or an ATP therapy site based on the cardiac sensed activity at the LV sensing sites, the ATP therapy module to control delivery of antitachycardia pacing (ATP) therapy at the ATP therapy site.

2. The device of claim 1, wherein the ATP therapy module delivers a stimulus to electrodes at one or more of an LV site, and right ventricular (RV) site, the detection module to sense evoked responses at the LV sensing sites, the ATP therapy module to designate the ATP therapy site to include at least the LV sensing site with a shortest activation time relative to the one or more LV site, and RV site where the stimulus is delivered.

3. The device of claim 1, wherein the ATP therapy module delivers a stimulus or stimuli at a stimulus site to the heart during VT and measures post pacing intervals (PPI) between the stimulus site and the LV sensing sites, the ATP therapy module to designate the ATP therapy site to include the LV sensing site having a shortest PPI.

4. The device of claim 1, wherein the ATP therapy module delivers a stimulus to the heart and obtains electrograms associated with the LV sensing sites, the ATP therapy module to determine a degree of fractionation of the electrograms, the ATP therapy module to designate the ATP therapy site to include the LV sensing site corresponding to a least degree of fractionation, the degree of fractionation being assessed based on at least one of a number of deflections in the electrograms, a number of peaks in the electrograms, a width of the electrograms, an area under the electrograms and a fast Fourier transform (FFT) of the electrograms.

5. The device of claim 1, further comprising an impedance module to measure impedance associated with the LV electrodes during normal sinus rhythm or a pacing therapy, the ATP therapy module to designate the ATP therapy site to include the LV sensing site having a minimum electrode impedance.

6. The device of claim 1, wherein the ATP therapy module determines an activation pattern of activation times corresponding to the LV sensing sites, the ATP therapy module designating the ATP therapy site to include the LV sensing site with an earliest activation time.

7. The device of claim 1, wherein the ATP therapy module determines an activation pattern for a ventricular tachycardia (VT) reentrant circuit, the ATP therapy module designating the ATP therapy site to include the LV electrode proximate to a starting site or reentrant activation pathway of the VT reentrant circuit.

8. The device of claim 1, wherein the ATP therapy module designates at least one ATP therapy site based on at least one of an activation time, post pacing interval, waveform morphology, electrode impedance, or activation pattern.

9. The device of claim 1, wherein the ATP therapy module designates an ATP cycle length to correspond to a predetermined percentage of a VT cycle length.

10. The device of claim 1, wherein the ATP therapy module changes at least one of a stimulus voltage and pulse width for subsequent ATP therapies when a prior attempted ATP therapy is not successful in converting an arrhythmia to a normal sinus rhythm.

11. A method for controlling anti-tachycardia pacing (ATP), comprising:
sensing signals from a lead including multiple LV electrodes representative of cardiac activity at multiple LV sensing sites;
detecting an arrhythmia that represents at least one of a tachycardia and fibrillation;
identifying at least one of an ATP configuration or an ATP therapy site based on a relation between the cardiac activity at the LV sensing sites; and
controlling delivery of ATP therapy at the ATP therapy site.

12. The method of claim 11, further comprising;
delivering a stimulus to electrodes at one or more of an LV site, and right ventricular (RV) site; sensing evoked responses at the LV sensing sites; and
designating the ATP therapy site to include at least the LV sensing site with a shortest activation time relative to the one or more LV site, and RV site.

13. The method of claim 11, further comprising:
delivering a stimulus at a stimulus site to the heart;
measuring post pacing intervals (PPI) between the stimulus site and the LV sensing sites; and
designating the ATP therapy site to include the LV sensing site having a shortest PPI.

14. The method of claim 11, further comprising:
delivering a stimulus to the heart;
obtaining electrograms associated with the LV sensing sites;
determining a degree of fractionation of the electrograms, the degree of fractionation being assessed based on at least one of a number of deflections in the electrograms, a number of peaks in the electrograms, a width of the electrograms, an area under the electrograms and a fast fourier transform (FFT) of the electrograms; and
designating the at least one of ATP configuration or the ATP therapy site to include the LV sensing site corresponding to a least degree of fractionation.

15. The method of claim 11, further comprising:
measuring impedance associated with the LV electrodes during normal sinus rhythm or a pacing therapy; and
designating the ATP therapy site to correspond to the LV sensing site having minimum electrode impedance.

16. The method of claim 11, further comprising determining an activation pattern of activation times corresponding to the LV sensing sites; and
designating the ATP therapy site to be the LV sensing site with an earliest activation time.

17. The method of claim 11, further comprising determining an activation pattern for a ventricular tachycardia (VT) reentrant circuit; and designating the ATP therapy site to be the LV electrode proximate to a starting site or reentrant path of the VT reentrant circuit.

18. The method of claim 11, further comprising designating at least one ATP therapy site based on at least one of an activation time, post pacing interval, waveform morphology, electrode impedance, or activation pattern.

19. The method of claim 11, further comprising designating an ATP cycle length to correspond to a predetermined percentage of a VT cycle length.

20. The method of claim 11, further comprising changing at least one of a stimulus voltage and pulse width for subsequent ATP therapies when a prior attempted ATP therapy is not successful in converting an arrhythmia to a normal sinus rhythm.
